(19) United States
(12) Patent Application Publication
(10) Pub. No.: US 2020/0400561 A1
(43) Pub. Date: Dec. 24, 2020

(54) METHOD FOR INSPECTING TEXTILE ARTICLES

(71) Applicant: Herbert Kannegiesser GmbH, Vlotho (DE)

(72) Inventors: Wilhelm Bringewatt, Porta Westfalica (DE); Engelbert Heinz, Vlotho (DE)

(73) Assignee: Herbert Kannegiesser GmbH, Vlotho (DE)

(21) Appl. No.: 16/970,135

(22) PCT Filed: Feb. 14, 2019
(86) PCT No.: PCT/EP2019/000042

§ 371 Date: Aug. 14, 2020

(30) Foreign Application Priority Data
Feb. 16, 2018 (DE) 10 2018 0011 92.6
Aug. 14, 2018 (DE) 10 2018 006 395.0
Sep. 11, 2018 (DE) 10 2018 007 184.8

(51) Int. Cl. G01N 21/3581; D06F 95/00; G01N 21/89; G01N 23/04; G01N 23/18; G01N 23/083; G01N 33/36

(52) U.S. Cl. CPC G01N 21/3581; D06F 95/00; G01N 21/89; G01N 23/04; G01N 2223/40; G01N 23/083; G01N 33/367; G01N 2223/04; G01N 23/18

(57) ABSTRACT

A method for inspecting work coats by x-raying, in which the x-ray radiation is shielded or reduced by x-raying in a chamber or spatially sufficiently remote from the persons so that the persons are not exposed to impermissibly high levels of radiation, for use in freeing used textile articles such as work coats from foreign bodies before being treated in laundries, which has previously been done manually, which is time-consuming and in itself entails a risk of injury to the persons freeing the work coats from foreign bodies.

### CROSS REFERENCE TO RELATED APPLICATIONS

This application is the US National Phase of and claims the benefit of and priority on International Application No. PCT/EP2019/000042 having a filing date of 14 Feb. 2019, which claims priority on and the benefit of German Patent Application No. 10 2018 001 192.6 having a filing date of 16 Feb. 2019 and German Patent Application No. 10 2018 006 395.0 having a filing date of 14 Aug. 2018.

### BACKGROUND OF THE INVENTION

### Technical Field

The invention relates to a method for inspecting textile articles, in particular used textile articles, possible foreign bodies in the textile articles being determined and/or subsequently separated from the textile articles, and/or a method for inspecting textile articles, in particular used textile articles, possible foreign bodies in or on the textile articles being determined.

### Prior Art

Textile articles, in particular used textile articles, such as laundry, for example clothing items, work clothing items, flat linen or the like, need to be inspected for foreign bodies before cleaning, in particular washing. The foreign bodies subsequently need to be removed from textile articles that have foreign bodies.

The inspection of the textile articles for foreign bodies and the removal thereof has to date been carried out manually. This is cost-intensive and requires a relatively large amount of time, so that the cycle time, in particular of the washing process, is unnecessarily prolonged.

### BRIEF SUMMARY OF THE INVENTION

The object of the invention is to provide a method for inspecting textile articles, in particular used textile articles, which allows the inspection of textile articles, in particular for the presence of foreign bodies, in an automated and time-saving fashion.

A method for achieving this object is a method for inspecting textile articles, in particular used textile articles, possible foreign bodies in the textile articles being determined and/or subsequently separated from the textile articles, characterized in that the determination and/or detection of the foreign bodies is carried out with electromagnetic rays at a position separated from persons such that a radiation exposure of the person lies below a maximum allowed limit value. According to this method, the determination of foreign bodies in or on the textile articles is carried out with electromagnetic rays having the same spectrum or different spectra. Very many more textile articles may therefore be automatically inspected per unit time than would be possible manually. Furthermore, the inspection of the textile articles is carried out with electromagnetic rays at a position separated and/or shielded from persons such that a radiation exposure of the person lies below a maximum allowed limit value. In this way, the inspection may be carried out with electromagnetic rays, for example X-rays or gamma rays, against which persons need to be protected.

Preferably, the inspection of the textile articles by detecting foreign bodies possibly present therein is carried out with electromagnetic rays that are suitable for making the foreign bodies, not only metallic foreign bodies but also other nonmetallic foreign bodies, in the textile articles visible. This may preferably be terahertz radiation, X-radiation or gamma radiation. Because possible foreign bodies in textile articles are made visible, presorting may be carried out according to textile articles without foreign bodies and textile articles that respectively have at least one foreign body. Only these textile articles then subsequently need to be provided for having the foreign bodies removed.

In one preferred configuration of the method, the textile articles are exposed in the state at least partially freely hanging down to the same or different foreign body-detecting electromagnetic radiation/radiations. Preferably, this is done in a state of the textile articles hanging from a continuous conveyor and/or hanging from or on a hanger. By the inspection of the textile articles in the at least partially hanging state, the textile articles are stretched or spread sufficiently for it to be possible to detect foreign bodies in and/or on them automatically by electromagnetic radiation.

Preferably, the detection of the foreign bodies is carried out on individual textile articles. Accordingly, each textile article per se is inspected individually for the presence of at least one foreign body. Depending on how great the throughput of textile articles to be inspected per unit time is, two or even more than two textile articles may be inspected simultaneously. This is preferably done by inspecting each textile article per se. It is preferably done using a plurality of devices for the production of the same electromagnetic radiation, or in particular of electromagnetic radiation differing in respect of the spectrum, and for detection.

According to one particularly advantageous possible configuration of the method, the detection of the foreign bodies in or on the textile articles is carried out in the region of a storage section for the textile articles. As an alternative or in addition, the detection of the foreign bodies may be carried out in a plane, in particular a level, which lies above or below a plane, or a level, on which persons handle and/or process the laundry items. Because of the inspection of the textile articles for foreign bodies in a different plane, or different height, the inspection of the textile articles takes place far away from manual work on the textile articles. This ensures that the persons work at a sufficient distance from the position at which the textile articles are inspected with electromagnetic rays. This distance is preferably so large that the persons are no longer subjected to any significant radiation exposure, but at least the radiation exposure lies below the maximum allowed limit value. This ensures that the electromagnetic rays, in particular terahertz radiation, X-radiation or gamma radiation, used for the inspection of the textile articles do not lead to any radiation exposure, or at least do not lead to any unallowably high radiation exposure, that could have a detrimental effect on persons.

The detection of possible foreign bodies in or on the textile articles preferably takes place after hanging them from the continuous conveyor or after hanging the textile article on the hanger or hanging from the hanger. By hanging the textile articles from the conveyor or hanging on hangers, in particular transport hangers, the textile articles hang down at least mostly from the conveyor or hanger. This favors and allows inspection with electromagnetic rays. By this inspection taking place after the hanging of the textile articles from the conveyor or hanging on transport hangers, the textile articles are examined for foreign bodies before subsequent processing operations, so that the foreign bodies found may be removed before subsequent treatment of the textile articles, in particular sorting, wet treatment or the like, takes place. In this way, the foreign bodies found and removed can no longer cause damage in the subsequent treatment of the textile articles and to the persons involved.

According to one possible refinement of the method, at least the foreign bodies made visible by the at least one electromagnetic radiation are recorded by at least one imaging device or at least one detector, which preferably image at least a part of the laundry item, and the data thereby obtained, preferably image data, of at least one image or at least one representation are stored in at least one electronic memory, for each individual laundry item. Preferably, only image data of those textile articles for which at least one foreign body has been made visible by the electromagnetic rays are stored. With the aid of the image data, it is possible to see how many and which foreign bodies there are in or on the respective textile article. Since the image data are preferably recorded from the flat side of the respective hanging textile article, the image data also provide knowledge about where the respective foreign body is in or on the textile article. With the aid of the information obtained, namely number, type and position of the foreign bodies, the respective foreign body may be separated or removed in a controlled and therefore time-saving fashion from the at least one textile article that has it. By storing one or each recorded image, for example X-ray image, of the respective textile article through which X-ray or similar electromagnetic rays have passed and/or which has been made transparent, and the foreign bodies located thereon or therein thereby being made visible, visualization or display of the foreign bodies is carried out in respect of their type, size and positioning on or in the respective textile article, preferably with allocation to the respective textile article.

By storage of the image data of one or more images, or recordings, of the respective textile article, these data may at any time be called up and/or compared with other image data. Thus, documentation may also be carried out and/or a database of typical foreign bodies may be produced with the aid of stored reference images. By comparing the foreign bodies stored in the database with current images, it is then possible to determine automatically by electronic means which type of foreign body is currently involved.

Preferably, the image data recorded by the at least one imaging device or at least one detector are visually represented as an image of the respective textile article with the at least one foreign body detected. This may, for example, be done by calling up the image data from the memory with the image data stored therein. The image data are visually represented at a position where there is no radiation exposure from the inspection process, or at least the radiation exposure lies below the maximum limit value. At least one person may then, without risk from the electromagnetic rays used for the inspection or visualization of foreign bodies, separate the foreign body or bodies in a controlled and rapid fashion from the respective textile article with the aid of the at least one image shown to him, and above all he cannot overlook or forget any foreign body. To this end, for example, the number of foreign bodies found in the respective textile article may additionally be displayed to the person as a number so that he knows accurately how many foreign bodies he needs to remove.

It is also conceivable to inspect the relevant article again after the removal of the at least one foreign body by the respective person, in order to check whether all foreign bodies have actually been removed by the person. This inspection is carried out in just the same way as described in the method above in connection with making foreign bodies visible. A separate device is preferably used for this, which may also be arranged at a different position, in particular a different level, where it is ensured that the persons working on the textile articles are not subjected to any radiation exposure, or at least are not subjected to a radiation exposure lying above the allowed limit value.

According to another advantageous possible configuration of the method, the textile articles, preferably in each case only a single textile article or only a few, for example two or three, textile articles are exposed to, or irradiated with, electromagnetic rays, in particular terahertz rays, X-rays and/or gamma rays, in an at least mostly closed chamber for the purpose of inspection. This chamber offers shielding of the electromagnetic radiation directed inside it onto the at least one textile article. No radiation, or at most reduced radiation, therefore emerges from the chamber. In this way, the inspection of the textile articles with electromagnetic rays may be carried out at a shorter distance from workplaces occupied by persons than would be possible in the case of inspecting the textile articles without such shielding in at least one chamber. As an alternative or optionally also in addition to the chamber, at least one beam source and/or at least one radiation generator may be shielded, for example by arranging it in a housing that—apart from the radiation exit—is shielded or shields.

According to one advantageous refinement of the method, the length of the textile articles, i.e. the distance between the upper end and the lower end, is reduced for the inspection with electromagnetic rays, in particular terahertz rays, X-rays or gamma rays. In this way, only a smaller area of the respective textile article needs electromagnetic radiation to be applied to it and/or electromagnetic radiation to pass through it. This reduces the required radiation dose. Preferably, the reduction of the length of the respective textile article is carried out at least in the region of the chamber in which the respective textile article is exposed to the electromagnetic radiation.

Furthermore, it is preferably conceivable to reduce the length of the respective textile article by a lower part of it being supported on a floor of the chamber or on a static guide surface and/or on a belt conveyor. The shortening of the length of the respective textile article then takes place so to speak by “compressing” at least a part of the article, a part, preferably a lower part, of the respective textile article being moved to the side and/or being pushed up.

According to one advantageous refinement of the method, an identification of the textile articles having at least one data carrier is carried out automatically before the inspection.

In particular, in the case of textile articles which are provided with a data carrier, the data carriers are read out before the inspection of these textile articles. By the data thereby acquired, information about the relevant textile articles to be inspected may be obtained. This preferably involves determining the type of the textile articles. It is also possible to derive therefrom whether the textile articles comprise something that could be regarded as a foreign body without actually being one, for example metal components, zip fasteners or buttons.

After the data carriers of the textile articles, specifically all textile articles or only textile articles that have data carriers, have been read out, the inspection of the textile articles is carried out. In this case, with the aid of the read-out data, a kind of categorization of the textile articles takes place. This categorization is carried out according to the type of the textile articles and/or the presence of objects, for example metallic and also nonmetallic buttons or zip fasteners, on the textile articles. During the evaluation of the result of the inspection, in particular of the foreign bodies thereby detected, the information found with the aid of the read-out data may be taken into account. If, for example, a row of nontextile objects lying above one another are found during the inspection, these may be ignored as foreign bodies because, with the aid of the previously read-out data of the data carrier of the respective textile article, it may be assumed that these are buttons that do not constitute foreign bodies.

A further method for achieving the object mentioned in the introduction, which may be an advantageous refinement of the method described above, is a method for inspecting textile articles, in particular used textile articles, possible foreign bodies in or on the textile articles being determined, characterized in that the inspection of the respective textile article for the presence of at least one foreign body is carried out with different spectra or different energy of the electromagnetic rays. In this method, the inspection of a respective textile article is carried out by electromagnetic rays with different spectra and/or different energy or intensity. This allows quantitative imaging of the textile articles through which radiation has passed, in particular quantitative X-ray imaging. Reliable detection of foreign bodies is thereby ensured. In particular, different nontextile materials may be determined and/or imaged.

Furthermore, a separate recording, in particular an image of the textile article through which radiation has passed, may be produced for each different spectrum with which the same textile article is inspected. By a comparison, in particular an automatic computer-assisted comparison, so to speak a “collation” of the different recordings or image data is carried out so that foreign bodies can be detected with high accuracy. In this way, foreign bodies of a very wide variety of types, in particular of different materials, for example metallic materials on the one hand and nonmetallic materials on the other hand, may be determined more reliably and preferably fully. Above all, in this way it is possible to detect foreign bodies that would not be found during an inspection with only one spectrum, in particular of X-rays but also of other electromagnetic radiation. As an alternative, it is conceivable to superpose the recordings of each spectrum of the same textile article and thereby produce a common image, i.e. so to speak a total image.

Preferably, the different spectra, in particular X-ray spectra, are produced simultaneously and recorded as an image and/or visually represented by separate detectors or only one detector having a plurality of detector regions, each for one spectrum. In this case, all spectra always pass through all textile articles and foreign bodies. With this procedure, different spectra do not need to be produced by the radiation generator. In the simplest case, a beam source having only a single spectrum is sufficient. The different spectra are then produced in front of or at the respective detector or detector region by at least one filter for the electromagnetic radiation, in particular X-radiation. Regions are thus formed with different spectra, which are recorded by the neighboring detector regions of a single detector or optionally also a plurality of detectors, each for one spectrum, and different recordings or images thereof are produced.

As an alternative, it is also conceivable to generate a plurality of electromagnetic radiations with different spectra, for example by a plurality of radiation generators. The respective textile article to be inspected is then exposed to electromagnetic radiation with different spectra. This is preferably done successively. Subsequently, the respective electromagnetic radiation is recorded by the at least one detector, preferably sequentially but optionally also simultaneously, for each different spectrum produced. In particular, a recording of each spectrum is made successively and these recordings are represented synchronously next to one another, for example to at least one person. This at least one person may then identify all foreign bodies by comparison of the images, and optionally different images.

In one preferred refinement of the method, it is however also conceivable to superpose the images electronically by a kind of collation, so that only one image on which all foreign bodies detected for the different spectra appear is produced. This allows particularly simple, rapid and reliable determination of all foreign bodies in or on the respectively inspected textile article, in particular by at least one person.

### BRIEF DESCRIPTION OF THE DRAWINGS

Preferred exemplary embodiments of the invention will be explained in more detail with the aid of the drawing, in which:

FIG. 1 shows a side view of a part of an apparatus having a detection device for textile articles;

FIG. 2 shows a central longitudinal section through the detection device of FIG. 1,

FIG. 3 shows a perspective view of the detection device of FIG. 2;

FIG. 4 shows a second exemplary embodiment of a part of an apparatus for inspecting textile articles in the region of the detection device; and

FIG. 5 shows an image, recorded by the detection device, of a spread-out textile article having three foreign bodies detected during the inspection.

### DETAILED DESCRIPTION OF PREFERRED EMBODIMENTS

The figures show various exemplary embodiments of a part, relating to the method according to the invention for inspecting textile articles, in particular shaped items and flat linen, of an apparatus.

The apparatus may be a transport system 10 for textile articles, which extends through at least a part of a laundry facility. In the present exemplary embodiment, the textile articles are shaped items, namely work coats 11, without the invention being intended to be restricted thereto. Accordingly, when only “work coats 11” are referred to below, this also includes other textile articles, i.e. it is to be understood as “work coats 11 and other textile articles”. A plurality of work coats 11, but also different textile articles, for example work coats 11, pants, T-shirts, shirts, jackets or flat linen may be transported simultaneously by the transport system 10, these being transported successively and/or following one another, preferably while being spaced apart, in the transport direction 12 through the laundry facility.

The transport system 10 has a preferably revolving, static conveyor line 13, for example having at least one rail. The conveyor line 13 has a profile corresponding to the desired transport path of the work coats 11 or the like through the laundry facility, and is preferably formed as a continuous conveyor.

Carriages or carriers, preferably spaced apart, may be movable on the conveyor line 13. The carriages or carriers are moved at least along selected portions of the conveyor line 13 by a revolvingly drivable drive line, for example a chain, assigned thereto, in the transport direction 12 along the conveyor line 13. This may be done continuously or discontinuously. Along certain portions of the conveyor line 13, the work coats 11 may be transportable further freely and by the force of gravity. To this end, such portions of the conveyor line 13 have a correspondingly inclined profile.

Transport hangers 15 are hung below the respective carriage or on the conveyor line 13. The transport hangers 15 may be permanently connected to the associated carriages, or may also be hung removably. In the exemplary embodiment shown, each transport hanger 15 has a hanger hook 16, a hanger housing 17 arranged at the lower end thereof, and preferably identical hanger arms 18 assigned to two opposite sides of the hanger housing 17. A work coat 11 may respectively be hung on the hanger arms 18 extending laterally from the hanger housing 17 on opposite sides so that the work coats 11 hang down from the transport hanger 15. The hanger arms 18 of the transport hanger 15 may be foldable for automatic unhanging of the work coats 11. It is also conceivable to hang the transport hangers 15 with their hanger hooks 16 on the conveyor line, which then does not need to have a carriage but only carriers.

In the exemplary embodiment shown, the transport hangers 15 also respectively have a clip 14, for example for pants or flat linen items, below their hanger housings 17.

At the start, the conveyor line 13 has a preferably horizontally extending loading portion 19 located in a lower plane. There, a work coat 11 may respectively be hung on a transport hanger 15 by at least one person 20. As seen in the transport direction 12, the loading portion 19 is followed by a rising section 21 of the conveyor line 13. Along the rising section 21, the work coats 11 hanging on the transport hangers 15 are transported up into a second, higher plane. In this second higher plane, there may be at least one storage area 22 for a multiplicity of work coats 11 hanging on transport hangers 15. At least in the region of the storage area 22, the work coats 11 are oriented transversely with respect to the transport direction in close succession, so that a multiplicity of work coats 11 can be accommodated in the storage area 22. In the region of the storage area 22, the conveyor line 13 preferably has a meandering profile with a plurality of preferably parallel portions, neighboring portions being connected at their ends to form the continuous conveyor line 13. The number of portions is dictated by the size of the storage area 22, i.e. the number of work coats 11 which the storage area 22 can contain. At the end of the storage area 22, the conveyor line 13 has a descending section 23 along which the work coats 11 pass from the higher plane in the region of the storage area 22 back into the lower plane in which the loading portion 19 is located. In this lower plane, the descending section 23 is followed by a preferably horizontal foreign body removal section 24. At the foreign body removal section 24, foreign bodies 37 may be removed manually from work coats 11 that have them by at least one further person 25.

In the region of the storage area 22 located in a higher level, optionally in front of or behind it, at least one detection device 26 is provided for inspecting the work coats 11 for at least one foreign body 37 located therein or thereon. The detection device 26 is configured for automatic contactless determination, in particular imaged recording and/or representation, of foreign bodies 37 in the work coats 11 by means of irradiation and/or radiation through the work coats 11 with electromagnetic rays. The electromagnetic rays are terahertz radiation, in particular X-radiation or possibly also gamma radiation. Only “X-raying” and “X-radiation” will be referred to below, without the invention thereby being intended to be restricted thereto.

In the exemplary embodiment shown, the detection device 26 is configured to inspect respectively at least one individual work coat 11 hanging from a transport hanger 15 of the transport system 10 for the presence of at least one foreign body 37. To this end, the detection device 26 has at least one beam source 27, which may also be referred to as a radiation source, for producing X-rays or similar electromagnetic rays for making foreign bodies 37 in or on the work coats 11 visible. The at least one beam source may be shielded. Furthermore, the detection device 26 has at least one detector 28 which records as an image and/or scans the textile article, in particular the work coat 11, or at least a part thereof which may usually have foreign bodies, during the X-raying thereof. The at least one detector 28 may therefore also be referred to as an X-ray scanner, which also detects, in particular records as an image and/or scans, foreign bodies 37 that may have been made or become visible during the X-raying. The detector 28 preferably has at least one line detector, or the detector 28 is at least one line detector, which produces line-by-line an image of the respective inspected work coat 11 and of at least one foreign body 37 possibly contained therein. As an alternative, the detector 28 may also be provided with a camera that records the image, produced by the beam source 27, of the work coat 11 with the possible one foreign body 37, and/or it may be configured as a camera.

In the exemplary embodiment shown, the detection device 26, i.e. the beam source 27 and the detector 28, which together in particular form an X-ray scanner, is arranged in a mostly closed chamber 29. The chamber 29 is configured, particularly in respect of its material, in such a way that it at least mostly prevents the emergence of the electromagnetic rays, in particular X-rays, gamma rays or other terahertz rays, produced by the beam source 27. In addition to the chamber 29, shielding of the at least one beam source 27 may be provided, although shielding may also replace the chamber 29.

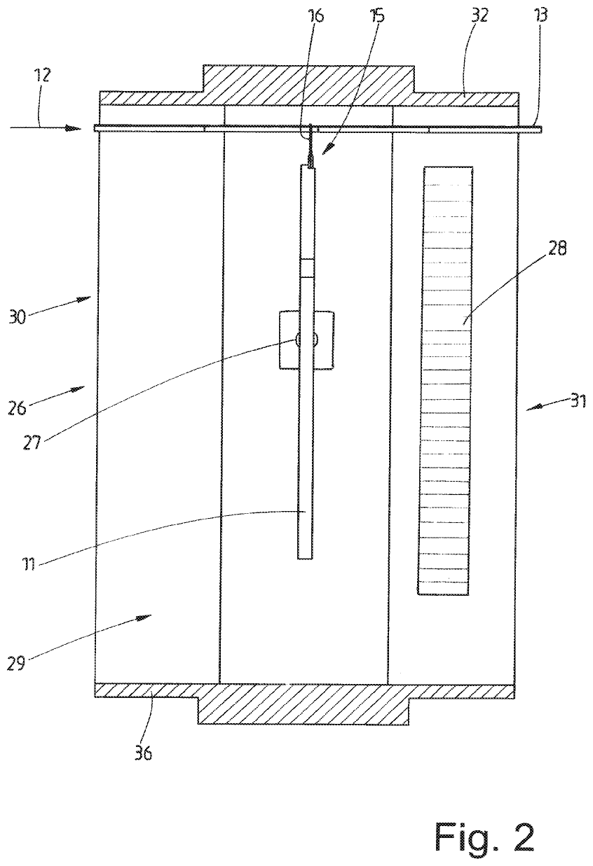

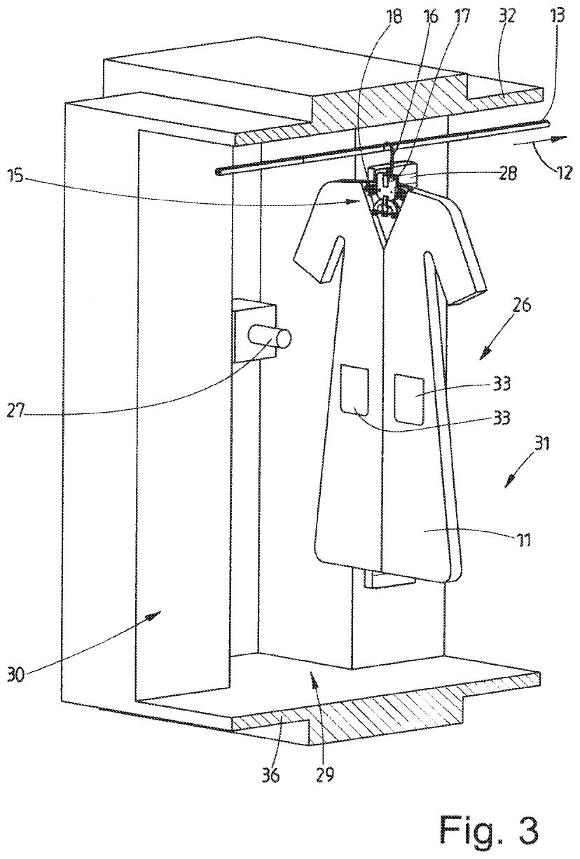

The chamber 29 is furthermore configured in such a way that the work coats 11 can be conveyed individually through it in succession by the transport system 10. To this end, the chamber 29 has an inlet opening 13 and an outlet opening 31, which may optionally be closable. The inlet opening 30 and the outlet opening 31 follows one another in the transport direction 12, i.e. they are arranged at the start and at the end of the chamber 29. In the exemplary embodiment shown, the work coats 11 are transported through the chamber 29 with an orientation extending transversely with respect to the transport direction 12. The transport direction of the transport system 10 therefore extends along the surface normals of the (large) flat sides of the work coats 11. So that the at least one detector 28, in particular the line detector represented in FIGS. 2 and 3, of the detection device 26 can then record as an image and/or scan a flat side with this orientation of the work coats 11, the detector 28 can be deployed and/or tilted from a resting position, shown in FIGS. 2 and 3, next to the work coat 11. The detector 28 can scan a flat side of the relevant work coat 11 and produce image data thereof. The detector 28 is moved only briefly to a distance in front of the flat side of the work coat 11 in order to produce an image of the work coat 11 with foreign bodies 37 made visible by X-raying, and then is immediately moved back into its starting position in order to allow the respective work coat 11 to be transported through the chamber 29. Likewise, the at least one beam source 27, preferably the X-ray source, can be deployed and/or tilted from a position shown in FIGS. 2 and 3 into a working position, in which it is directed onto the flat side, facing away from the detector 28, of the work coat 11. As an alternative, it is conceivable to arrange the detector 28, in particular the line detector, obliquely with respect to the transport direction 12, for example at an angle of 45°, next to the transport path of the work coat 11 through the chamber 29. The detector 28 does not then need to be moved forward and back in order to record an image of the X-rayed work coat 11.

Furthermore, it is also conceivable to transport the work coat 11 through the chamber 29 with an orientation extending transversely with respect to the transport direction 12. Movement, or an oblique orientation, of the detector 28 is then also obviated because the latter then always views, or is directed at, a flat side of work coat 11.

In the exemplary embodiments shown, the conveyor line 13 of the transport system 10 is guided through the chamber 29, specifically close below along the roof 32 thereof. In the region of the chamber 29, the conveyor line 13 then extends through its optionally closable inlet opening 30 and outlet opening 31 together with the respective work coat 11 and the transport hanger 15 carrying the latter. It is, however, also conceivable to guide the conveyor line 13 of the transport system 10 away over the roof 32 of the chamber 29. The conveyor line 13 then extends outside the chamber 29. The hanger hook 16 of the transport hanger 15 in this case extends through the roof 32 of the chamber 29. To this end, the roof 32 of the chamber 29 would need to be provided with a narrow slot which is continuous in the transport direction 12.

Arranged inside the chamber 29 are the at least one beam source 27 and the detector 28 opposite the latter, which is configured here as a vertical bar-like line detector. The work coats 11 to be inspected pass through the chamber 29 at the detector 28, so that the respective work coat 11 can be exposed to electromagnetic rays, in particular X-rays, of the beam source 27. The detector 28, in particular the line detector, is arranged or movable relative to the respective work coat 11 in such a way that a flat side of the work coat 11 can at least mostly be scanned in order to produce, in particular, an X-ray scan of work coat 11. The at least one detector 28 is dimensioned and arranged in such a way that it does not irradiate and/or influence the transport system 10 and/or the transport hanger 15, but only at most the respective textile article.

The image data recorded by the detector 28 are preferably stored in an image file. In particular, an identifying feature, for example a number, may be assigned to the work coat 11 or another textile article.

In contrast to the representation in FIG. 2, it is conceivable to arrange the detector 28 configured as at least one line detector in a horizontal longitudinal extent. Such a line detector may be shorter than in the exemplary embodiment of FIG. 2. The line detector is preferably moved up and/or down in front of a flat side of the textile article on a vertical path, surface scanning of the textile article being carried out. It may be sufficient to move the horizontal line detector up and down in front of, or optionally also behind the work coat 11 to be detected, or another textile article, only to such an extent that the part or region of the textile article in which there may typically be foreign bodies is scanned.

The respective detector 28, in particular the vertical line detector (FIG. 2) or the horizontal line detector, may be arranged “gliding”. This is expediently done in such a way that at least the at least one detector 28, and optionally the entire detector device 26, is moved along synchronously with the work coats 11 being transported further by the transport system in the transport direction 12. This allows detection of foreign bodies during the further transport of the work coats 11 or other textile articles. The detection of foreign bodies may thus be carried out during continuous further transport of the textile articles, in particular work coats 11.

According to another conceivable alternative exemplary embodiment of the apparatus, the detection of foreign bodies in or on the textile articles is carried out on a transport band not affected by the beams of the at least one detection device 26. The textile articles are then inspected while lying flat on the transport band by exposure to electromagnetic radiation. The transport band configured in the manner of a belt conveyor successively transports textile articles lying flat thereon past the detection device 26. With this apparatus, continuous detection of the textile articles in respect of foreign bodies is possible. Furthermore, the textile articles lying flat on the transport band may be inspected, in particular continuously scanned, very advantageously and above all surface-wide for foreign bodies with electromagnetic rays.

At the foreign body removal section 24, the person 25 working there is assigned a monitor 34. On the monitor 34, the image data recorded by the detection device 26, in particular the X-ray scan, is displayed visually to the operator 25 for the respective work coat 11 currently moving past him. Preferably, only image data of work coats 11 being transported past the person 25 that have at least one foreign body 37 are displayed. Work coats 11 for which the detection device 26 has not found any foreign bodies 37 are able to be transported continuously past the person 25 in the region of the foreign body removal section 24.

The distance from the detection device 26 to the persons closest to it, in particular the persons 20 and 25, is selected in such a way that they are not subjected to any significant radiation exposure, and in particular are subjected at most to a radiation exposure below the maximum allowed limit value. This is achieved by arranging the detection device 26, preferably together with the storage area 22, on a different—in this case higher—plane or level. In addition, as in the exemplary embodiment shown, or also as an alternative, the radiation exposure of the person is further reduced to a minimum by arranging the detection device 26 in a chamber 29 used as shielding. Optionally, there may also be an intermediate ceiling (not shown in the figures) between the upper and lower levels. When such an intermediate ceiling is provided, it is conceivable to omit the chamber 29 because the intermediate ceiling then fulfills the function of shielding. In the case of shielding of the electromagnetic radiation by the intermediate ceiling and/or the chamber 29, persons, in particular the persons 20 and 25, may be spatially positioned without risk closer to the detection device 26, in particular its beam source 27, in order to do their work there.

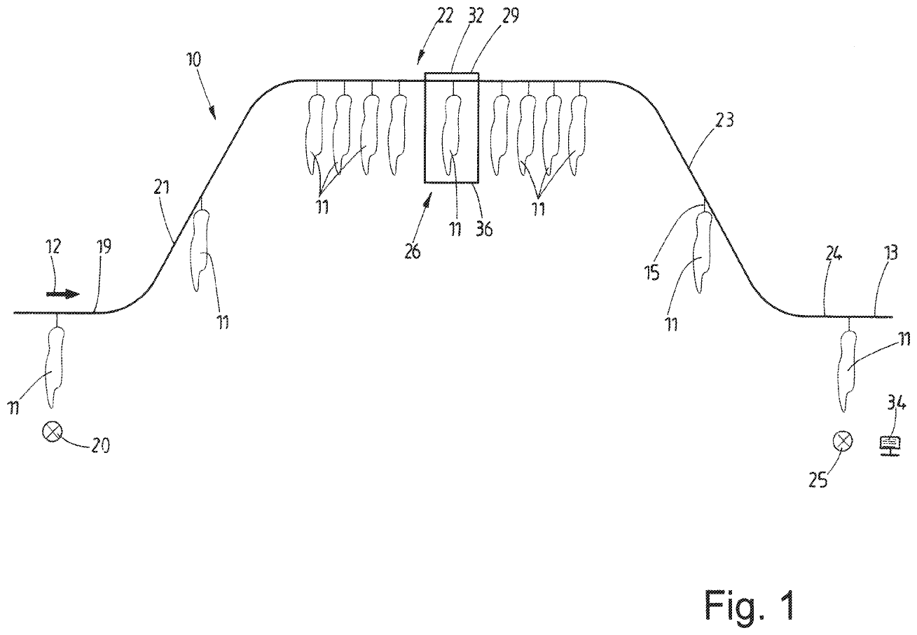

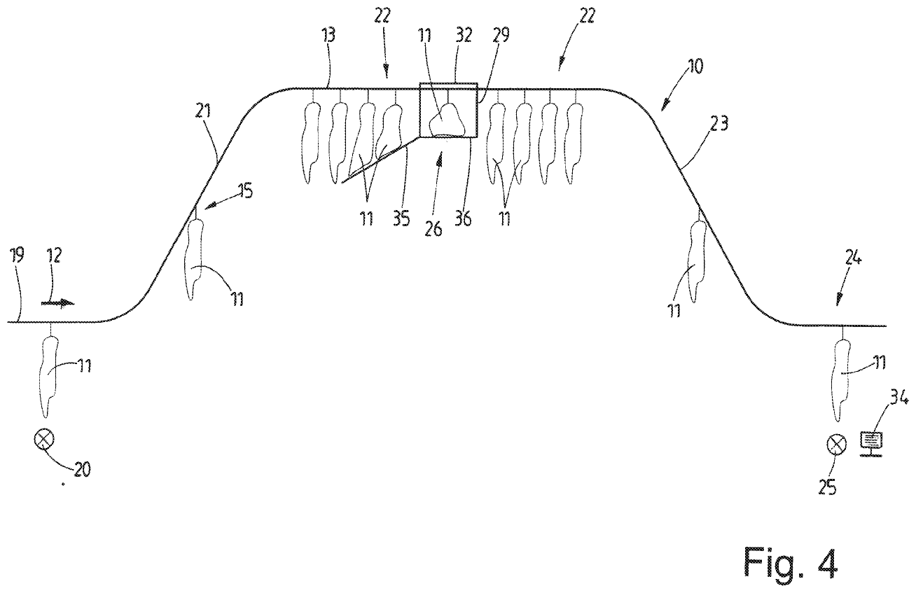

FIG. 4 shows an alternative exemplary embodiment of the apparatus of FIG. 1. In this exemplary embodiment, the chamber 29 is assigned a static guide surface 35 for lower regions of the work coats 11. As seen in the transport direction 12, this guide surface 35 before chamber 29 has an obliquely rising initial portion which, shortly before the inlet opening 30 of the chamber 29, joins with a horizontal portion. This horizontal portion may be arranged on or above a floor 36 of the chamber 29. It is, however, also conceivable for this horizontal portion of the guide surface 35 to be formed by the floor 36 of the chamber 29. By the portion of the guide surface 35 rising obliquely as seen in the transport direction 12, a lower part of the work coat 11 is turned back when sliding along the guide surface 35 and the length of the work coat 11 is thereby reduced, so that the volume of the chamber 29 may be reduced and have a height that is less than the lengths of the work coats 11.

As an alternative, instead of the guide surface 35, it is also possible to provide a revolving belt conveyor, the profile of which corresponds to the profile of the guide surface 35. The folded or turned-back lower ends of the work coats 11 are then not pulled along while sliding over the guide surface 35 but are transported further by the belt conveyor without slipping, or only with reduced slipping, synchronously or almost synchronously during the further transport of the work coat 11 by the transport system 10.

The method according to the invention will be explained by way of example below with the aid of the apparatus described above:

The textile articles represented in the figures as work coats 11, but also ones that are not work coats 11, are inspected with electromagnetic rays for the presence of foreign bodies 37 at a sufficient distance from persons carrying out tasks, in particular the persons 20 and 25. In the exemplary embodiment shown, this is done by scanning the work coats 11 with X-rays (X-ray scan). Other electromagnetic rays may also be used for the determination of foreign bodies 37 in or on the work coats 11, for example terahertz radiation or gamma radiation. The inspection of the work coats 11 with electromagnetic radiation is carried out at a distance from the person working closest to the detection device 26 used therefor, in particular the persons 20 and 25, such that they are not subjected to any significant radiation exposure. At least, however, the radiation exposure of the persons 20, 25 is reduced and/or shielded to such an extent that it lies below established in particular statutory, limit values and/or maximum values.

In the exemplary embodiment shown, the work coats 11 are successively examined individually for the presence of foreign bodies 37 while hanging on transport hangers 15. This examination is carried out by the detection device 26 in the region of a storage area 22 for a multiplicity of work coats 11 hanging on transport hangers 15. This storage area 22 is located in a different plane, in the exemplary embodiment shown a higher plane, in particular a level above the plane or level on which at least one person 20 hangs work coats 11 on the transport hangers 15 and/or the foreign bodies 37 are removed by at least one operator 25 from the X-rayed textile articles, for example work coats 11, after their detection.

By the detection device 26, which has at least one beam source 27, preferably an X-radiation source, and at least one detector 28, in particular a line detector, an individual work coat 11 is respectively irradiated or radiated through, preferably X-rayed, and possible foreign bodies 37 are thereby made visible, and an image of the work coat 11 with foreign bodies 37 possibly present therein is produced and/or recorded by the detector 28. The detector 28 therefore delivers image data of the respectively inspected work coat 11. These image data are preferably assigned to the associated work coat 11 in an image file or stored as an image and preferably visually displayed, in particular as an image and/or an X-ray image, to at least one person 25 at the foreign body removal section 24.

In the apparatuses shown in FIGS. 1 and 4, the detection devices 26 are arranged in a chamber 29. This chamber is used to shield the X-rays or equivalently acting electromagnetic rays produced by the beam source 27. It also contributes to persons, in particular persons 20 and 25, who are working closest to the detection device 26 with the beam source 27 not being subjected to significant radiation exposure, or at least the radiation exposure lies below the established limit values. If the detection device 26 with the beam source 27 is arranged in the chamber 29 used for shielding, the storage area 22, or its storage section, and in particular the detection device 26, do not need to be arranged in a different plane or level. The detection device 26 with the beam source 27 may then be located in the same level as that in which persons are working, in particular the persons 20 and 25 working closest to the beam source.

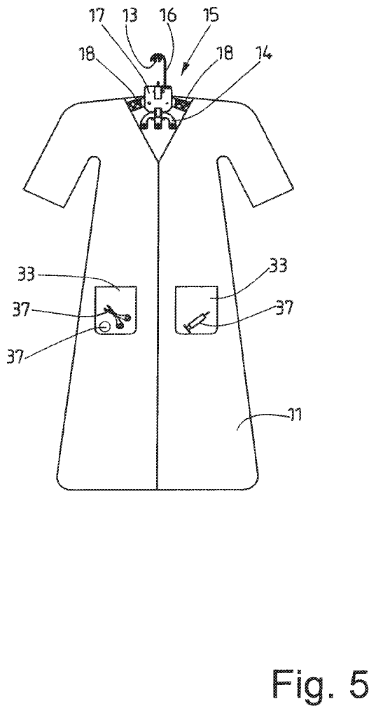

FIG. 5 schematically shows an image, in particular an X-ray scan, produced and recorded by the detection device 26. It shows the contours of the work coat 11 together with its pockets 33. Because of the X-raying of the work coat 11 with electromagnetic radiation by the detection device 26, foreign bodies 37 in the work coat 11 are also made visible. By way of example, a coin, a pair of scissors and a syringe are represented as foreign bodies 37 in the pockets 33 in FIG. 4. Other foreign bodies may likewise be determined by the detection device 26, for example writing implements, as well as ones which are not located in the pockets 33 of the work coat 11 but, for example, are attached in the collar region. Name badges or the like may also possibly be detected as foreign bodies 37 and recorded as an image by the detection device 26.

After leaving the storage area 22, the work coats 11 inspected by the detection device 26 for the presence of foreign bodies 37 enter the region of the foreign body removal section 24. Work coats 11 in which no foreign bodies 37 have been found are transported continuously through the foreign body removal section 24, for example to a sorting device or a laundry treatment device. Work coats 11 having at least one foreign body 37 are stopped close to the person 25 in the region of the foreign body removal section 24, so that they remain there briefly in order for the person 25 to be able to remove all foreign bodies 37 from or out of the work coat 11. The person 25 at the foreign body removal section 24 is assisted during the separation of the foreign bodies 37 from the work coat 11 by the image determined by the detection device 26 (FIG. 5), which for this purpose is called up from the image database and displayed on the monitor 34 while being assigned to the work coat 11 currently located in front of the person 25. The image that belongs to the work coat 11 currently stopped in the vicinity of the person 25 is always displayed on the monitor 34. With the aid of the image on the monitor 34, the person 25 can see how many foreign bodies 37 there are on or in the work coat 11, where the foreign bodies 37 are, for example in which pocket 33, and/or which type of foreign body 37 is involved. If, for example, a syringe is detected as a foreign body 37, the person 25 knows that he must be careful not to injure himself when taking it out of the pocket of the work coat 11.

It is conceivable that the number of foreign bodies 37 which are in the work coat 11 currently located in front of the person 25 is additionally displayed to him on the monitor 34. The person 25 then knows how many foreign bodies 37 need to be removed from the respective work coat 11. Optionally, the person 25 may need to confirm the removal of each foreign body 37 on the monitor 34, or on a keypad assigned thereto. If, for example, there are three foreign bodies 37 in the work coat 11 (as in FIG. 5), the work coat 11 is not transported further until the person 25 has confirmed three times the removal of, in each case, one foreign body 37 from the pockets 33 of the work coat 11.

Possible alternative configurations of the invention, in which a plurality of work coats 11 are inspected simultaneously, may be envisioned. Each detection device 26 then has a number of detectors 28 at least equal to the number of work coats 11 being detected simultaneously. It may be sufficient, even in the case of a plurality of work coats 11 to be inspected simultaneously, for the detection device 26 to have only one radiation source 27. It is likewise conceivable, in the case of a plurality of work coats 11 subjected simultaneously to the X-ray scan, for the detection device 26 to have a plurality of X-ray sources or other beam sources 27.

The invention has been described above in connection with work coats 11. The invention is not, however, restricted thereto. It is suitable for inspecting any type of textile articles for foreign bodies 37, in particular any clothing items (shaped items) and also flat linen (table linen, bed linen or the like).

One alternative exemplary embodiment of the invention, which preferably supplements and/or refines the exemplary embodiment described above, relates to textile articles which have at least one data carrier. The data carriers may be ones that can be contactlessly read out optically, optoelectronically or purely electromagnetically. Such data carriers may be adhesive labels, barcodes, QR codes and/or data memories, for example RFID chips or NFC chips.

Before the inspection of the textile articles, the data carriers of each textile article, or at least of those textile articles which have data carriers, are read out contactlessly individually.

With the data coming from the relevant laundry item, more detailed information about the textile article and/or the laundry item may be obtained, in particular information which could be of interest for the subsequent inspection of the textile article in respect of possible foreign bodies. Preferably, the type of a textile article which is involved, for example a work coat 11, is determined from the determined or recorded data of the respective textile article.

From the knowledge obtained, categorization is carried out before the actual inspection of the textile articles, for example according to whether the textile articles to be inspected typically have metallic components, and what these are, for example buttons or zip fasteners, and according to those textile articles which usually do not have buttons, zip fasteners or other components because of their type.

The determination of all or some data stored on their data carriers, carried out before the inspection of the textile articles, leads to a reliable, in particular error-free, inspection of foreign bodies by electromagnetic rays. If, for example, it is found from the data of a textile article that it is a work coat 11 with metallic or nonmetallic buttons, although these are detected by the electromagnetic rays during the inspection, because of the arrangement, position and/or shape of the buttons on the work coat 11 such buttons may be distinguished from other foreign bodies which have been detected together with the buttons during the inspection by electromagnetic rays. The buttons are then not detected as foreign bodies, or are ignored during the evaluation of the inspection. If, however, when reading out the data of a textile article it is found that it is a T-shirt, which does not have any additional components, any object detected during the subsequent inspection with electromagnetic rays represents a foreign body.

In the exemplary embodiment described above, which preferably supplements the first exemplary embodiment described in the introduction, at least almost error-free detection of foreign bodies is possible by applying electromagnetic rays to the textile article to be checked, since, because of the previously read-out data carrier of the respective textile article, the type thereof is known and in this way the textile article is therefore identified. During the evaluation of the result of the inspection by electromagnetic rays, metallic and preferably also nonmetallic objects, for example buttons and zip fasteners, which the article respectively to be inspected has, are not detected and/or represented as foreign bodies.

Another exemplary embodiment of the invention, which may preferably be implemented in combination with the exemplary embodiments described above, but which may also be implemented independently thereof, is distinguished in that the respective textile article, preferably each textile article, is inspected with electromagnetic rays, in particular X-rays, having a plurality of different spectra in order to determine the foreign bodies therein or thereon. Preferably, for electromagnetic rays, in particular X-rays, of each different spectrum, determination of the foreign bodies is carried out by producing a recording of the same textile article with each of the different spectra, and subsequently preferably superposing and/or collating the recordings and producing a single common image therefrom.

Preferably, the respective textile article is inspected by means of only one X-radiation, or other electromagnetic radiation, having only a single spectrum, or a particular constant spectrum. A single beam source, which only produces one particular spectrum, or X-ray spectrum, is then sufficient in order to inspect the textile articles. The respective textile article is then exposed to X-radiation of the same spectrum. However, this X-radiation is then not delivered directly to at least one detector. Instead, the at least one detector is preceded by or assigned a means which at least partially modifies the spectrum of the X-radiation. This means attenuates at least a part of the spectrum of the X-radiation coming from the beam source. The spectral composition of the preferably single spectrum, produced by the beam source, of the electromagnetic radiation is thereby modified. In this way, X-rays with different spectra and/or different spectral compositions may be formed behind the means. The means for modifying a part of the uniform spectrum coming from the radiation source is preferably configured as a filter which is suitable for modifying the X-radiation, or other electromagnetic radiation, in respect of its spectrum.

The X-rays transmitted with different spectra and/or different spectral compositions by the filter are preferably recorded by a single detector which has separate detector regions, specifically a separate detector region for each of the different spectra. As an alternative, however, it is also conceivable for a plurality of separate detectors to be provided behind the at least one filter in the radiation direction. Here, a separate detector is then provided for each spectrum and/or each spectral composition.

The method according to the exemplary embodiment described above will be explained below with the aid of an example with X-rays, the spectrum of which has been attenuated:

The preferably single beam source, in particular a single X-ray source, produces X-radiation of one spectrum. After passing through the textile article or reflection, this X-radiation strikes the at least one detector, which is preferably assigned to the side of the textile article to be inspected facing away from, or remote from, the X-ray source. Before the at least one detector, the X-ray spectrum is at least partially modified. From the preferably uniform radiation transmitted through the respective textile article, or reflected by it, two different X-ray spectra are therefore formed, each having a different spectral composition.

The two different X-ray spectra are imaged through at least one filter provided in front of or at the respective detector. From the filter, X-rays with two different spectra and/or different compositions then travel to the detector, which in the case of two mutually different spectra per se is formed with two detector regions. This detector evaluates the different spectral compositions synchronously, in particular simultaneously, so that two different recordings and/or image data sets of the textile article respectively to be inspected are formed. These are then electronically collated and/or superposed in order to form a single image, visible to a person, of the X-rayed textile article. This image simultaneously contains both foreign bodies that have been imaged by one spectrum and foreign bodies that have been imaged by the second spectrum. With the aid of an X-ray image of the inspected textile article, the person may then identify all foreign objects therein and thereon, specifically foreign bodies of different materials, above all materials that are not textile. The materials that are not textile need not necessarily be metallic materials, and they may also be metallic materials on the one hand and nonmetallic materials on the other hand, i.e. foreign bodies such as for example plastic buttons, handkerchiefs, in particular paper tissues, plastic zip fasteners or the like.

In the case of foreign bodies made partly of plastic and partly metal, for example, density information of plastic materials and metallic materials may for example be obtained on the basis of different spectra. By combining and/or collating the recordings, images or image data produced from different spectra, a single image, which provides the observer with additional information about the material or the materials of the respective foreign body, is then produced for the observer. For example, syringes having a plastic or glass body and a metallic cannula may be fully identified as such for the observer.

### LIST OF REFERENCES

10 transport system
11 work coat
12 transport direction
13 conveyor line
14 clip
15 transport hanger
16 hanger hook
17 hanger housing
18 hanger arm
19 loading portion
20 person
21 rising section
22 storage area
23 descending section
24 foreign body removal section
25 person
26 detection device
27 beam source
28 detector
29 chamber
30 inlet opening
31 outlet opening
32 roof
33 pocket
34 monitor
35 guide surface
36 floor
37 foreign body

1. A method for inspecting textile articles, in particular used textile articles, possible foreign bodies (37) in the textile articles being determined and/or subsequently separated from the textile articles, comprising carrying out the determination and/or detection of the foreign bodies (37) with electromagnetic rays at a position separated from persons (20, 25) such that a radiation exposure of the persons (20, 25) lies below a maximum allowed limit value.

2. The method as claimed in claim 1, wherein the detection of the foreign bodies (37) is carried out with electromagnetic radiation that makes the foreign bodies (37) in the textile article visible.

3. The method as claimed in claim 1, wherein the textile articles are exposed to electromagnetic radiation, which detects the foreign bodies (37) or makes them visible, in the at least partially hanging state, preferably a state hanging from a transport device (10) and/or hanging from or on a transport hanger (15).

4. The method as claimed in claim 1, wherein the detection of the foreign bodies (37) is carried out on an individual textile article, preferably during continuous further transport thereof.

5. The method as claimed in claim 1, wherein the detection of the foreign bodies (37) is carried out in the region of a storage section and/or a storage area (22) for the textile articles, and/or the detection of the foreign bodies (37) is carried out in a level which lies above a level in which the persons (20, 25) handle or process the textile articles.

6. The method as claimed in claim 1, wherein the detection or inspection of the relevant textile article in respect of foreign bodies (37) is carried out after the hanging of the respective textile article from the transport device (10), preferably after the hanging of the respective textile article on the transport hanger (15) or the hanging of the textile article from the transport hanger (15).

7. The method as claimed in claim 2, wherein at least the foreign bodies (37) made visible by the electromagnetic radiation are recorded by at least one imaging device and/or a detector (28), which preferably also image at least a part of the textile article, and data thereby obtained are stored in a memory, particularly in an image memory, for each individual textile article, preferably only such a textile article in which at least one foreign body (37) has been determined.

8. The method as claimed in claim 7, wherein the data recorded by the at least one imaging device and/or detector (28) are visually represented to at least one of the persons (25), preferably at a position with a radiation exposure below the maximum allowed limit value, as an image of the textile article with the at least one foreign body (37) detected and/or made visible by the electromagnetic rays.

9. The method as claimed in claim 1, wherein the textile articles, preferably only in each case an individual textile article or only a few textile articles, are exposed to the electromagnetic rays in a chamber (29), in particular an at least mostly closed chamber (29) and/or a chamber (29) shielded against the emergence of at least a majority of the electromagnetic rays.

10. The method as claimed in claim 9, wherein the length of the textile articles is reduced for the inspection with the electromagnetic rays, preferably reduced in the chamber (29), in particular with a lower part of the respective textile article being supported on a floor (36) of the chamber (29) or on a guide surface (35) arranged on or above the floor (36) or on a belt conveyor, at least in the chamber (29).

11. The method as claimed in claim 1, further comprising carrying out an identification and/or classification of the textile articles before the inspection of the textile articles.

12. The method as claimed in claim 1, data carriers of the textile articles are read out and the type of the respective textile article is determined or derived therefrom before the inspection of the textile articles with the electromagnetic rays, and/or an individual evaluation, assessment and/or identification of the result of the inspection of a respective one of the textile articles, preferably of the foreign bodies detected during the inspection, is carried out with the aid of the read-out data or the determined type of the respective textile article, in particular as a function of the previously determined type of the respective textile article.

13. A method for inspecting textile articles, in particular used textile articles, possible foreign bodies (37) in or on the textile articles being determined, comprising carrying out the inspection of a respective textile article for the presence of at least one foreign body (37) with different spectra or different energy of electromagnetic radiation at a position separated from persons (20, 25) such that a radiation exposure of the persons (20, 25) lies below a maximum allowed limit value.

14. The method as claimed in claim 13, wherein at least one recording is produced for all spectra of the electromagnetic radiation from the same textile article.

15. The method as claimed in claim 13, wherein the determination of foreign bodies (37) is carried out in or on the respective textile article by evaluation, in particular comparison, superposition and/or collation, of all recordings of the textile article to be inspected made with different spectra.

16. The method as claimed in claim 13, characterized in that the different spectra of electromagnetic rays are produced simultaneously and/or the electromagnetic rays of different spectra are recorded by at least one detector having a plurality of detector regions, in particular one detector region for each spectrum, the plurality of detection regions preferably being formed by different filters or different filter regions of a filter for modifying the spectrum of the electromagnetic radiation.

17. The method as claimed in claim 16, characterized in that each of the different spectra of the electromagnetic rays is recorded by its own detection region or detector by the at least one detector having a plurality of different detection regions, and/or in the case of a plurality of detectors, each of the different spectra of the electromagnetic rays is recorded by its own detector.
